(19) United States
(12) Patent Application Publication
(10) Pub. No.: US 2026/0308379 A1
(43) Pub. Date: Oct. 8, 2026

(54) THERMOPLASTIC COLLAGEN COMPOSITIONS COMPRISING POLYESTERS

(71) Applicant: VISCOFAN ESPAÑA, S.L.U., TAJONAR (ES)

(72) Inventors: LORENZO LIZCANO DE VEGA, TAJONAR (ES); LLUCIÀ CORDERO CERRADA, TAJONAR (ES); CARLOS MARÍA LONGO ARESO, TAJONAR (ES); JESÚS MARIA IZCO ZARATIEGUI, TAJONAR (ES); MONICA GAMARRA VAZQUEZ, TAJONAR (ES)

(21) Appl. No.: 19/484,614

(22) PCT Filed: May 16, 2024
(86) PCT No.: PCT/EP2024/063530

§ 371 Date: Nov. 14, 2025

(30) Foreign Application Priority Data
May 17, 2023 (EP) 23382462.2

(51) Int. Cl. C08L 89/06; C08J 5/18; C08K 5/053; C08L 67/02; C08L 67/04; C09D 7/63; C09D 167/02; C09D 167/04; C09D 189/06; C12N 5/00

(52) U.S. Cl. CPC C08L 89/06; C08J 5/18; C08L 67/02; C08L 67/04; C09D 7/63; C09D 167/02; C09D 167/04; C09D 189/06; C12N 5/0018; C08J 2367/02; C08J 2367/04; C08J 2389/06; C08J 2467/02; C08J 2467/04; C08J 2489/06; C08K 5/053; C08L 2201/06; C08L 2203/16; C08L 2205/06; C12N 2533/54

(57) ABSTRACT

Blend compositions including: i) collagen, ii) water, iii) particularly a plasticizer, and iv) a polyester having a melting point equal to or lower than 120° C. The weight ratio of the collagen and the polyester in the blend composition is from 2:1 to 1:20. The blend composition provides a sheet having a water solubility equal to or lower than 40% w/w; and a homogeneous film when submitted to a blown film extrusion. The blend composition and corresponding films can be used to make solid articles, particularly those for use in the food industry, in the pet food sector, in the agricultural sector, or in the pharmaceutical sector.

This application claims the priority of the European Patent Application 23382462.2 filed on May 17, 2023.

The invention relates to the field of collagen-based materials, particularly, to thermoplastic collagen compositions comprising a thermoplastic collagen and a polyester. These compositions have adequate mechanical properties to conform them into a variety of solid shaped articles.

### BACKGROUND ART

Collagen is the most abundant extracellular matrix protein of animals, representing around 30% of the whole protein content. It is mostly found in connective tissue such as cartilage, bones, tendons, ligaments, and skin. Up to 28 different types of collagens have been described in the literature. The basic structural unit of collagen is a heterotrimer consisting of three helical protein chains (known as alpha chains), which are twisted among each other in form of a triple helix. These molecules are ordered in a parallel and staggered way forming fibrils and fibers.

Collagen is one of the proteins which has been applied most successfully in industry. It is used, among other fields, in food industry (e.g., as a film forming material in packaging) or in the pet food sector (e.g., chewing bones or functional food), in the agricultural sector, as well as for pharmaceutical or medical purposes (e.g., as biocompatible implants or as carriers for the cultivation of cells in in vitro tests). The biodegradability of collagen is of great advantage with regard to the waste management after use.

Collagen containing tissues can be used as raw materials in the preparation for the different industrial products. The technologies which lead to these products make use of collagen at different stages of processing and degradation, ranging from intact undenatured collagen fibers to highly degraded gelatin. For some applications, one of the disadvantages of gelatin is its high water solubility which is the consequence of the fragmentation of protein chains by hydrolyzation during the denaturation process and damage of cross-links in the collagen structure.

As a biopolymer, collagen can be processed by thermoplastic techniques, such as extrusion or injection molding, in order to obtain articles of different forms including cast or molded pieces, sheets, films, or coatings, in analogy to synthetic polymers. Scaffolds made of natural biopolymers such as collagen have the advantage of their biocompatibility and biodegradability. For such processes intact undenatured collagen fibers or highly degraded gelatin are generally not suitable. In order to use collagen as a thermoplastically processable biopolymer, a partially denatured collagen which maintains long protein chains but reduces their cross-linking and intertwining must be used. Such a collagen can be designated as thermoplastic collagen (TC). It is produced from collagenous tissues, such as bovine or porcine hide, and includes a denaturation step. After production is mixed with water. Other additives, such as glycerin, dyes or inorganic salts may be optionally added. Extrusion or injection molding of thermoplastic collagen is operated at moderate temperatures (90-100° C.) and results in versatile products, such as pellets, threads, sheets, films, or molded parts.

Thermoplastic collagen has been described in the prior art. For example, document EP2727938 discloses a process for obtaining collagen from animal skin which includes the processing of the skin in mixing cylinders by mechanical grinding treatment at 50-70° C. The physical properties of the collagen material thus obtained make it susceptible of being transformed by thermoplastic transformation techniques. However, in the hands of the present inventors it was found that thermoplastic collagen obtained according to this document showed high solubility almost comparable to gelatin. Besides, depending on how many days had passed from its production, blown film extrusion made from the thermoplastic collagen disclosed in EP2727938 was either not possible or resulted in films that showed inhomogeneity, high thickness variation and extremely high film tackiness. Additionally, it was found that such thermoplastic collagen showed a pronounced melt flow rate (MFR) decrease after 7 or after 23 days.

Further, document WO2007104322 (Naturin) discloses a denatured or partially denatured dry collagen powder based on a fibril forming collagen and its use for preparing thermoplastic collagen-based compositions comprising it together with water and optionally a plasticizer. However, in the hands of the present inventors blown extrusion of monolayer films made from such thermoplastic collagen-based compositions was problematic and the quality of sample was not acceptable; films had uneven thickness, showing very rough surface with visible granules in the film. Additionally, the films became very brittle after 24 or 48 hours.

Thus, from what is known in the state of the art and despite the advances made in the field, there is still the need to provide thermoplastic collagen compositions with improved mechanical properties, which may be used as highly customizable scaffolds.

### SUMMARY OF INVENTION

The inventors have found that when a polyester having a melting point equal to or lower than 120° C. is added to a thermoplastic collagen made from a mixture of collagen, water, and particularly a plasticizer, a blend composition is obtained which shows adequate mechanical properties to conform them into a variety of solid shaped articles such as complex films, mono-oriented filaments, extrusion blown bottles, 3D printed or injection molded objects. In particular, the blend composition of the invention is able to provide homogeneous films by blown film extrusion with no visible granules, and also shows a reduced solubility in comparison to gelatin or the thermoplastic collagen disclosed in the prior art without the presence of the polyester.

As it is demonstrated in the experimental section of the present application, the collagen-based blend compositions of the invention are able to provide materials which show a number of technical advantages listed below:
Lower variability of melt flow rate (melt fluidity) over time
Good processability in thermoforming processes
Lower dependency of mechanical properties on ambient relative humidity
Lower water solubility, i.e., higher water resistance
Lower oxygen and water vapour permeation (OTR, WVTR)
Good processability in 3D printing (Fused deposition modelling, FDM)
Significant increase in tensile strength of single oriented (yarn) specimens
Improved adhesion and thus cell viability with respect to polyester.

Thus, a first aspect of the invention related to a collagen-based blend composition comprising a blend composition comprising:
i) collagen;
ii) water;
iii) particularly a plasticizer; and
iv) a polyester having a melting point equal to or lower than 120° C.;
wherein the weight ratio of the collagen and the polyester is from 2:1 to 1:20.

A second aspect of the invention relates to a solid shaped article which is obtainable by a process comprising:
a) melt blending the collagen-based blend composition as defined herein; and
b) extruding, casting or blowing the blend of step a) into a solid article.

In addition, the examples also illustrate that the collagen-containing blend compositions of the invention are able to provide films obtained by platen press with improved stability. It was found that, samples containing thermoplastic collagen in the absence of a polyester do not withstand cell culture conditions (37° C. in culture medium) because the collagen is dissolved after a short time, whereas this does not occur when the thermoplastic collagen is mixed with a polyester such as PCL. Thus, it has been found that by varying the ratio of collagen to polyester, the degradation and reabsorption rate can be modulated; the higher the ratio of collagen to polyester the lower the time of degradation and reabsorption, and viceversa. This confers the blend composition of the invention interesting properties to be used in implants and in cell cultures.

Accordingly, a third aspect of the invention relates to the use of the blend composition comprising a collagen and polyester of the first aspect as support for in vitro assays.

A further aspect of the invention relates to the use of the collagen-based blend composition or the solid shaped article as defined herein, in the food industry, in the pet food industry, in the agricultural industry, or in the pharmaceutical industry.

### BRIEF DESCRIPTION OF THE FIGURES

FIG. 1 shows the cell viability (% V) of different films. As a positive control C+, cells were seeded onto standard commercial polystyrene well plates treated for optimal cell attachment (Corning ref 3595). Film 1 corresponds to a Collagen Cell Carrier® membrane (CCC) produced by Viscofan, Films 2 and 3 correspond to films according to the present invention (Film 2: CF30+PCL (30:70), Film 3: CF30+PCL (70:30)), and Film 4 corresponds to 100% PCL).

FIG. 2 shows pictures of materials printed by a domoBIO 2A (Domotek, Spain) printer with the blends according to the invention comprising CF30+PCL (70:30), (50:50), and (30:70).

### DETAILED DESCRIPTION OF THE INVENTION

All terms as used herein in this application, unless otherwise stated, shall be understood in their ordinary meaning as known in the art. Other more specific terms as used in the present application are as set forth below and are intended to apply uniformly throughout the specification and claims unless an otherwise expressly set out definition provides a broader definition.

For the purposes of the present invention, all given ranges include both the lower and the upper endpoints of the range. Ranges given, such as temperatures, times, weights, and the like, should be considered approximate, unless specifically stated.

The term “about” or “around” or “approximately” as used herein refers to a range of values±10% of a specified value. For example, the expression “about 10” or “around 10” includes ±10% of 10, i.e., from 9 to 11.

The collagen-based blend composition of the invention is thermoplastic. For the purposes of the invention, the term “thermoplastic” refers to a plastic polymer material or composition that becomes pliable or mouldable at a certain elevated temperature and solidifies upon cooling. A thermoplastic is capable of being repeatedly softened by heating and hardened by cooling. The term “thermoplastic collagen” as used herein refers to a partially denatured collagen produced from collagenous tissues, such as bovine or porcine hide, which is always mixed with water and particularly a plasticizer, and which shows thermoplastic properties.

The term “plasticizer” as used herein refers to a substance capable to produce or promote plasticity when added.

The term “biodegradable” as used herein refers to a composite or product capable of being broken down (e.g. metabolized and/or hydrolyzed into innocuous degradation products) over time in the environment of use.

The term “biocompatible” as used herein refers to a material which does not provoke an adverse response in a living being. For example, a suitable biocompatible material when introduced into the body of a human subject does not itself provoke a significant immune response and is not toxic to the subject.

The terms “molecular weight”, “average molecular weight” and “Mw” have the same meaning and are used herein interchangeably. The molecular weight is calculated by the following equation:

$${M}_{w} = \frac{{\sum }_{i} {N}_{i} {M}_{i}^{2}}{{\sum }_{i} {N}_{i} {M}_{i}}$$

where Ni is the number of molecules of molecular mass Mi. The mass average molecular mass can be determined by light scattering, size exclusion chromatography (SEC), and sedimentation velocity.

For the purposes of the invention, the term “melt flow rate” (MFR), also known as melt flow index (MFI), refers to a measure of the ease of flow of a polymer melt. It is defined as the mass of polymer, in grams, flowing in ten minutes through a capillary of a specific diameter and length by a pressure applied via prescribed alternative gravimetric weights at alternative prescribed temperatures. The MFR variation percentage after n days is calculated by the formula:

$$MFR variation ( \% ) = ( MFR at n days - MFR at 0 days ) / ( MFR at 0 days ) \times 100$$

wherein MRF values are measured in g/10 min as detailed in the examples.

The term “oxygen transmission rate” (OTR) refers to the rate at which oxygen is transmitted through a film, when measured according to the method set forth in the examples.

The term “water vapor transmission rate” (WVTR) as used herein refers to the rate at which water vapor is transmitted through a film, when measured according to the method set forth in the examples.

The term “tensile strength at break” refers to the tensile stress at the moment at which a test sample breaks. Tensile stress is the force placed on the test sample divided by the cross-sectional area of the sample.

The expression “obtainable by” is used herein for defining a product (e.g., the blend composition or the solid shaped article) by its preparation process and refers to the product that can be obtained through the preparation process disclosed herein. For the purposes of the invention, the expressions “obtainable”, “obtained” and similar equivalent expressions are used interchangeably and, in any case, the expression “obtainable” encompasses the expression “obtained”.

A first aspect of the invention relates to a blend composition comprising i) collagen, ii) water, iii) particularly a plasticizer, and iv) a polyester having a melting point equal to or lower than 120° C.

In one embodiment, optionally in combination with one or more features of the various embodiments described above or below throughout all the description, the blend composition is meltable. For the purposes of the invention, the term “meltable” means that the blend composition upon heating to its melt temperature, becomes molten and/or flowable, and when cooled to below its melting temperature, it solidifies.

In another embodiment, optionally in combination with one or more features of the various embodiments described above or below throughout all the description, the blend composition is in solid state at room temperature (20-25° C.), more particularly is in solid state at a temperature equal to or lower than 40° C. Even more particularly, the solid is in form of solid pellets.

In another embodiment, optionally in combination with one or more features of the various embodiments described above or below throughout all the description, the blend composition is in solid state at a temperature equal to or lower than 40° C. and is meltable.

In another embodiment, optionally in combination with one or more features of the various embodiments described above or below throughout all the description, the blend composition is able to be thermoformed, in particular by melt blending the collagen-based blend composition as defined herein; and extruding, casting or blowing the blend of step a) into a solid article.

In another embodiment, optionally in combination with one or more features of the various embodiments described above or below throughout all the description, the collagen of the blend composition is partially denatured. For the purposes of the present invention, the term “partially denatured” refers to a collagen with a degree of denaturation from 80 to 95% measured by Differential Scanning calorimetry (DSC) by rehydrating a collagen sample overnight with water; introducing the rehydrated product in a DSC vessel and sealing it and registering the DSC (heating index of 5 K/min). From the relative areas below the peak near 60° C. (typically observed for a completely native collagen and absent for a completely denatured collagen) an evaluation can be made regarding the degree of denaturation of the collagen sample.

In another particular embodiment, optionally in combination with one or more features of the various embodiments described above or below throughout all the description, water is present in an amount from 5 to 40%, more particularly from 5 to 35%, even more particularly from 7 to 30%, even more particularly from 7 to 25%, even more particularly from 7 to 15%, by weight with respect to the total weight of the blend composition. In another embodiment, optionally in combination with one or more features of the various embodiments described above or below, water is present in the composition in an amount about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, or about 40% by weight with respect to the total weight of the blend composition.

The collagen-based blend composition of the invention may optionally comprise a plasticizer. According to one embodiment, optionally in combination with one or more features of the various embodiments described above or below throughout all the description, the collagen-based blend composition comprises a plasticizer.

In one particular embodiment, optionally in combination with one or more features of the various embodiments described above or below throughout all the description, the weight ratio of the collagen and the plasticizer is from 100:5 to 100:60, more particularly from 100:10 to 100:40, even more particularly from 100:10 to 100:30. In another embodiment, optionally in combination with one or more features of the various embodiments described above or below, the plasticizer is present in the composition in an amount such that the weight ratio of plasticizer to collagen is about 100:5, about 100:10, about 100:15, about 100:20, about 100:25, about 100:30, about 100:35, about 100:40, about 100:45, about 100:50, about 100:55, or about 100:60.

In another particular embodiment, optionally in combination with one or more features of the various embodiments described above or below throughout all the description, the plasticizer is selected from the group consisting of glycerin, ethylene glycol, a mono-, di-, or triester of glycerin with carboxylic acids such as (C₃-C₁₂) alkanoic acids, sulfated fatty acids, lecithin, xylitol, sorbitol, ethylene glycol, 1,2-propyleneglycol, diethylene glycol, triethylene glycol, polyethylene glycol (e.g. 200 to 10000 Da), polypropylene glycol, ethylene diglycol, propylene diglycol, ethylene triglycol, propylene triglycol, polyethylene glycol, polypropylene glycol, 1,2-propanediol, 1,3-propanediol, 1,2-butanediol, 1,3-butanediol, 1,4-butanediol, 1,5-pentanediol, 1,6-hexanediol, 1,5-hexanediol, 1,2,6-hexanetriol, 1,3,5-hexanetriol, neopentyl glycol, trimethylol propane, pentaerythritol, inositol, mannitol, triethanolamine, sorbitol ethoxylate, glycerin ethoxylate, pentaerythritol ethoxylate, sorbitol acetate, pentaerythritol acetate, formamide, acetamide, N,N-dimethylformamide, N,N-dimethylacetamide, and mixtures thereof.

Non-limiting examples of sulfated fatty acids include sulfated castor oil, sulfated olive oil, sulfated soybean oil, sulfated sunflower oil and the like. Non-limiting examples of tri(C₃-C₁₂)alkylesters of glycerin include glycerol triacetate, glycerol tributyrate, glycerol trioleate, glycerol tripalmitate, glycerol tristearate, and the like.

In another particular embodiment, optionally in combination with one or more features of the various embodiments described above or below throughout all the description, the collagen-based blend composition comprises a plasticizer which is selected from the group consisting of glycerin, ethylene glycol, a tri(C₃-C₁₂)alkylester of glycerin, a sulfated fatty acid, lecithin, and a combination thereof, more particularly the plasticizer is glycerin, even more particularly the glycerin is present in an amount such that the weight ratio of glycerin to collagen is from 5:100 to 60:100, more particularly from 10:100 to 40:100, even more particularly from 10:100 to 30:100.

The collagen-based blend composition of the invention further comprises a polyester having a melting point equal to or lower than 120° C. The use of polyesters having this melting point has the advantage that the processability temperature of the blend composition to transform it into other materials such as films can be reduced in comparison to polyesters having a higher melting point and thus, the presence of water vapor can be avoided. As a result, homogeneous films with low roughness may be obtained. By contrast, when polyesters having a melting point higher than 120° C., for example polylactic acid (PLA), (see table 11) it was not possible to extrude any film.

There is no limitation on the type of polyester that can be used provided that it has a melting point equal to or lower than 120° C., in particular at 101300 Pa.

In another embodiment, optionally in combination with one or more features of the various embodiments described above or below, the polyester has a melting point equal to or lower than 115° C., equal to or lower than 110° C., equal to or lower than 100° C., equal to or lower than 90° C., equal to or lower than 80° C., equal to or lower than 70° C., equal to or lower than 60° C., equal to or lower than 55° C., in particular at 101300 Pa.

In another embodiment, optionally in combination with one or more features of the various embodiments described above or below, the polyester has a melting point equal to or higher than 50° C., equal to or higher than 60° C., equal to or higher than 70° C., equal to or higher than 80° C., equal to or higher than 85° C., in particular at 101300 Pa.

According to another embodiment, optionally in combination with one or more features of the various embodiments described above or below, the polyester is selected from the group consisting of polycaprolactone (PCL), polybutylene succinate-co-adipate (PBSA), polyethyleneadipate (PEA), polybutylenesuccinate (PBS) or polybutylene adipate terephthalate (PBAT), and combinations thereof. More particularly, the polyester is polycaprolactone (PCL) or polybutylene succinate-co-adipate (PBSA).

According to one embodiment, optionally in combination with one or more features of the various embodiments described above or below, the weight ratio of the thermoplastic collagen and the polyester is from 1.3:1 to 1:18. According to another embodiment, optionally in combination with one or more features of the various embodiments described above or below, the weight ratio of the thermoplastic collagen and the polyester is from 1:1.2 to 1:18. In another embodiment, optionally in combination with one or more features of the various embodiments described above or below, the weight ratio of the thermoplastic collagen and the polyester is about 2:1, about 1.9:1, about 1.8:1, about 1.7:1, about 1.6:1, about 1.5:1, about 1.4:1, about 1.3:1, about 1.2:1, about 1.1:1, about 1:1, about 1:1.5, about 1:1.6, about 1:1.7, about 1:1.8, about 1:1.9, about 1:2, about 1:3, about 1:4, about 1:5, about 1:6, about 1:7, about 1:8, about 1:9, about 1:10, about 1:11, about 1:12, about 1:13, about 1:14, about 1:15, about 1:16, about 1:17, about 1:18, about 1:19, or about 1:20.

According to one embodiment, the collagen-based blend composition comprises:
a) collagen;
b) water;
c) a plasticizer, particularly selected from the group consisting of glycerin, ethylene glycol, a tri(C₃-C₁₂)alkylester of glycerin, a sulfated fatty acid, lecithin, and a combination thereof, more particularly the plasticizer is glycerin;
b) a polyester having a melting point equal to or lower than 120° C., particularly polycaprolactone (PCL) or polybutylene succinate-co-adipate (PBSA); wherein the weight ratio of the collagen and the polyester is from 2:1 to 1:20, more particularly from 1.9:1 to 1:18, even more particularly from 1.3:1 to 1:18, and even more particularly from 1:1.2 to 1:18. More particularly, in the above embodiment, the weight ratio of the collagen and the plasticizer is from 100:5 to 100:60, more particularly from 100:10 to 100:40, even more particularly from 100:10 to 100:30.

Further, the combination of the collagen-based blend composition of the invention with other proteins may further provide additional process improvements or modification of mechanical properties such as tensile properties. Thus, in one embodiment, optionally in combination with one or more features of the various embodiments described above or below, the collagen-based blend composition further comprises soy protein or gluten.

In another embodiment, optionally in combination with one or more features of the various embodiments described above or below, the collagen-based blend composition is in the form of pellets.

It also forms part of the present invention a process for the preparation of the collagen-based blend composition as defined herein which comprises the steps of:
a) mixing a thermoplastic collagen which comprises collagen, water and particularly a plasticizer; and a polyester; and
b) melt blending and extruding the mixture of step a).

The invention also relates to a collagen-based blend composition as defined herein, which is obtainable by a process comprising the steps of:
a) mixing a thermoplastic collagen which comprises collagen, water and particularly a plasticizer; and a polyester; and
b) melt blending and extruding the mixture of step a).

It also forms part of the present invention a process for the preparation of the collagen-based blend composition as defined herein which comprises:
a′) premixing a collagen powder, water and particularly a plasticizer;
b′) mixing the composition of step a) and a polyester; and
c′) melt blending and extruding the mixture of step b′).

The invention also relates to a collagen-based blend composition as defined herein, which is obtainable by a process comprising the steps of:
a′) premixing a collagen powder, water and particularly a plasticizer to provide a mixture;
b′) mixing the mixture of step a) and a polyester; and
c′) melt blending and extruding the mixture of step b′).

Particularly, in the above embodiments, the weight ratio of the composition of step a) or alternatively step a′) and the polyester is from 75:25 to 5:95, more particularly from 70:30 to 10:90. Particularly, in the above embodiments, the weight ratio of the collagen and the polyester is from 2:1 to 1:20, more particularly from 1.9:1 to 1:18.

In another embodiment, optionally in combination with one or more features of the various embodiments described above or below, the thermoplastic collagen of step a) comprises i) from 40 to 75%, more particularly from 50 to 70%, even more particularly from 55 to 65% by weight of collagen,
ii) from 10 to 40%, more particularly from 15 to 35%, even more particularly from 20 to 30% by weight of water; and
iii) from 5 to 50% more particularly from 5 to 35%, even more particularly from 5 to 20% by weight of a plasticizer;
wherein the weight percentages are expressed with respect to the total thermoplastic collagen weight, and with the proviso that the sum of all the components of the thermoplastic collagen is 100%.

The thermoplastic collagen of step a) is commercially available (Ekomat CF10 and CF30, Ekolber SL). Alternatively, it can be prepared by methods well-known in the art, for example as disclosed in EP2727938.

In one embodiment, optionally in combination with one or more features of the various embodiments described above or below, the thermoplastic collagen of step a) is the one as disclosed in EP2727938, in particular in Example 1, but adjusting if needed the amounts of water and plasticizer.

In another embodiment, optionally in combination with one or more features of the various embodiments described above or below, the thermoplastic collagen and the polyester used in step a) are in the form of pellets.

In one embodiment, optionally in combination with one or more features of the various embodiments described above or below, the mixture of step a′) comprises from i) from 40 to 75%, more particularly from 45 to 55% by weight of collagen,
ii) from 10 to 40%, more particularly from 30 to 40% by weight of water; and
iii) from 5 to 50% more particularly from 10 to 20% by weight of a plasticizer; wherein the weight percentages are expressed with respect to the total weight of the mixture of step a′), and with the proviso that the sum of all the components of the mixture of step a′) is 100%.

The collagen of step a′) is commercially available (Kapro B95 SF, DCP). Alternatively, it can be prepared by methods well-known in the art, for example as disclosed in WO2007104322.

In one embodiment, optionally in combination with one or more features of the various embodiments described above or below, the collagen powder used in step a′) is the one disclosed in WO2007104322. More particularly, the collagen powder used in step a′) is a dry collagen powder being based on a fibril forming collagen being denatured or partially denatured, presenting an average molecular weight of at least 500 kDa, a solubility equal to or greater than 25% in water at 60° C. and an average particle size comprised between 30 μm and 350 μm.

In another embodiment, optionally in combination with one or more features of the various embodiments described above or below, the polyester used in step b′) is in the form of pellets.

In another embodiment, optionally in combination with one or more features of the various embodiments described above or below, step b′) is carried out in a mixer, particularly a laboratory z-blade mixer, for a suitable time and rpm conditions, for example during 5 minutes at 100 rpm.

In another embodiment, optionally in combination with one or more features of the various embodiments described above or below, the melt blending and extruding step b) or c′) is carried out in a co-rotating twin-screw extruder at an actual melt temperature, measured by any of temperature gauges of the twin screw extruder, equal to or lower than 120-130° C. For example, a co-rotating Twin-Screw-Extruder Leistritz GL27: D=27 mm, L/D=36 at 100 rpm may be used with a setting extrusion temperature from feeding zone to die head 50° C. to 100° C. at 4 kg/h to obtain at die exit two strands of 3 mm diameter which were cooled before being pelletized.

As mentioned above, the collagen-based blend composition of the invention is able to provide a sheet having a water solubility equal to or lower than 40% w/w. In one embodiment, optionally in combination with one or more features of the various embodiments described above or below, the water solubility is equal to or lower than 35% w/w, equal to or lower than 30% w/w, equal to or lower than 25% w/w, equal to or lower than 20% w/w, equal to or lower than 15% w/w, equal to or lower than 10% w/w.

The water solubility may be determined by:
a) providing a 1 mm sheet from the blend composition comprising i) collagen, ii) water, iii) particularly a plasticizer, and iv) a polyester having a melting point equal to or lower than 120° C.; at about 95° C. by means of a platen press;
b) cutting the obtained sheet into pieces of approx. 0.5-2 cm in width and length;
c) drying the sheet in a vacuum oven at about 160° C. until constant weight;
d) taking about 5 g of sample from the sheet and measuring its initial weight (Wᵢ);
e) soaking the sample of step c) into 100 ml of water under stirring for about 1 hour at about 60° C.;
f) separating and collecting the insoluble soaked sheet;
g) drying the soaked sheet in a vacuum oven at about 160° C. until constant weight;
h) measuring the final weight (Wₜ) of the sheet of step g); and
i) applying the following formula:

$$Water solubility ( \% ) = ( {W}_{i} - {W}_{f} ) / {W}_{i} * 100.$$

Additionally, the collagen-based blend composition of the invention is able to provide a homogeneous film when submitted to blow film extrusion.

More particularly wherein the extrusion is carried out in an extruder at an actual melt temperature, measured by any of temperature gauges of the extruder, which is about +15° C. higher than melting point of polyester and lower than 135° C.

For the purposes of the invention, the term “homogeneous film” refers to the fact that no visible granules are observed. A way to assess the presence or absence of visible granules is by measuring the roughness of the surface. In one embodiment, optionally in combination with one or more features of the various embodiments described above or below, the blown extruded film has a low roughness. The roughness may be determined as indicated in the examples below.

A further aspect of the invention relates to a solid shaped article which is obtainable by a process comprising:
a) melt blending the collagen-based blend composition as defined herein; and
b) extruding, casting or blowing the blend of step a) into a solid article.

According to one embodiment, optionally in combination with one or more features of the various embodiments described above or below, the solid shaped article is a film, an injection moulding article, a blow moulding article, a monofilament, a mono-oriented filament, an extrusion blown bottle, a 3D printed object, an injection molded object or a machined piece.

The films according to the invention may have a single layer (monolayer films) or may have two or more layers (multilayer films). Thus, in one embodiment, optionally in combination with one or more features of the various embodiments described above or below, the solid shaped article is a film, more particularly a film selected from a monolayer film or a multilayer film.

In one more particular embodiment, optionally in combination with one or more features of the various embodiments described above or below, the film is a mono-oriented film, more particularly a longitudinally oriented from 2.5 to 6 times.

The multilayer thermoplastic films according to the invention may have from 2 to 13 layers, particularly from 2 to 11, more preferably from 2 to 9, and even more preferably from 2 to 7 layers. In a particular embodiment, optionally in combination with one or more features of the various embodiments described above or below, the multilayer thermoplastic film has 3 or 5 layers.

The multilayer films may comprise one or more layers comprising caseinates as disclosed in the patent document EP2596051B1.

In one more particular embodiment, optionally in combination with one or more features of the various embodiments described above or below, the film is a monolayer film comprising the blend composition as defined herein optionally further comprising soy protein or gluten.

In another more particular embodiment, optionally in combination with one or more features of the various embodiments described above or below, the film is a multilayer film, wherein at least one of the layers of the multilayer film comprises the blend composition as defined herein, optionally further comprising soy protein or gluten, and the remaining layers comprise a polymer and optionally a compatibilizer.

In one embodiment, optionally in combination with one or more features of the various embodiments described above or below, the film is a multilayer film comprising at least three layers having A//B//C structure, wherein layer B comprises: i) the collagen-based blend composition as defined herein which optionally further comprises soy protein or gluten, and ii) optionally a compatibilizer; and layers A and C comprise a polymer, and optionally a compatibilizer.

In one particular embodiment, the polymer in the above embodiments is selected from the group consisting of polyethylene (PE), polypropylene (PP), polycaprolactone (PCL), polyethyleneadipate (PEA), polybutylene succinate-co-adipate (PBSA), polyhydroxyalkanoate (PHA), polyglycolide (PGA), polylactide (PLA), polylactide-co-glycolide (PLGA), polydioxanone (PDO), polybutylenesuccinate (PBS), polyethyleneterephtalate (PET), polytrimethyleneterephtalate (PTT), and polybutyleneterephtalate (PBT), polybutylene adipate terephthalate (PBAT), maleic anhydride grafted polyolefins (PE-g-MAH, PP-g-MAH), polyamide (PA), thermoplastic starch (TPS), thermoplastic cellulose, Ethylene Vinyl Alcohol (EVOH), polyvinyl alcohol (PVOH), and combinations thereof. More particularly, the polymer is selected from the group consisting of polyethylene (PE), polycaprolactone (PCL), polybutylene succinate-co-adipate (PBSA) and combinations thereof.

In another particular embodiment, the compatibilizer in the above embodiments is selected from the group consisting of Ethylene Vinyl Acetate (EVA), maleic anhydride grafted onto ethylene vinyl acetate copolymer (EVA-g-MAH), Polyvinyl acetate (PVAc), Vinyl Acetate Ethylene copolymer (VAE), Ethylene Ethyl Acrylate copolymer (EEA), Ethylene Methyl Acrylate copolymer (EMA), and combinations thereof.

In one particular embodiment, in the above embodiments layers A and C are the same. More particularly, layer A and C comprise a polymer selected from the groups consisting of polyethylene (PE), polycaprolactone (PCL), polybutylene succinate-co-adipate (PBSA), and combinations thereof.

In another particular embodiment, in the above embodiments layers A and C are different. More particularly, each of layer A and C independently comprises polycaprolactone (PCL) or polybutylene succinate-co-adipate (PBSA).

In another embodiment, optionally in combination with one or more features of the various embodiments described above or below, the film is a multilayer film comprising at least five layers having A//D//B//D//A structure, wherein layer B comprises: i) the collagen-based blend composition as defined herein which optionally further comprises soy protein or gluten, and ii) optionally a compatibilizer; layers A comprise a polymer as defined above; and layers D comprise a compatibilizer as defined above.

In one embodiment, optionally in combination with one or more features of the various embodiments described above or below throughout all the description, the films of the invention have a thickness from 10 to 200 μm, more particularly from 20 to 100 μm, as measured by scanning electron microscope (SEM).

According to one embodiment, optionally in combination with one or more features of the various embodiments described above or below, the solid shaped article is selected from the group consisting of an injection moulding article, a blow moulding article, a machine part and a filament for 3D printing by Fused Deposition Modelling (FDM); and comprises the collagen-based blend composition as defined herein which optionally further comprises soy protein or gluten, and optionally a compatibilizer.

In another embodiment, optionally in combination with one or more features of the various embodiments described above or below, the solid shaped article is a bottle or another 3-D multilayer article comprising at least three layers having A//B//C structure, wherein layer B comprises the collagen-based blend composition as defined herein which optionally further comprises soy protein or gluten; and layers A and C comprise a polymer as defined above.

In another embodiment, optionally in combination with one or more features of the various embodiments described above or below, the solid shaped article is a bottle or another 3-D multilayer article comprising at least five layers having A/D/B/D/A structure, wherein layer B comprises the collagen-based blend composition as defined herein which optionally further comprises soy protein or gluten; and layers A and C comprise a polymer as defined above.

In another embodiment, optionally in combination with one or more features of the various embodiments described above or below, the solid shaped article is a film and has one or more of the following properties:
a) a melt flow rate variation at 90° C. and load of 9.6 kg is equal to or lower than −50% after 7 days;
b) a melt flow rate variation at 120° C. and load of 9.6 kg is equal to or lower than −50 after 23 days;
c) a melt flow rate variation at 90° C. and load of 9.6 kg is from equal to or lower than −30% after 7 days;
d) a melt flow rate variation at 120° C. and load of 9.6 kg is from equal to or lower than −40% after 23 days;
e) an Oxygen Transmission Rate (OTR) equal to or lower than 600 cm³/m²·day, at 23° C. and 50% relative humidity;
f) a Water Vapor Transmission Rate (WVTR) equal to or lower than 800 g/m²·day, at 23° C. and 85% relative humidity;
g) a tensile strength at break in machine direction from 3 to 50 mPa;
h) a tensile strength at break in transverse direction from 3 to 30 mPa;
i) a tensile elongation at break in machine direction from 9 to 450%; and
j) a tensile elongation at break in transverse direction from 10 to 150%.

Throughout the description and claims the word “comprise” and variations of the word, are not intended to exclude other technical features, additives, components, or steps. Furthermore, the word “comprise” encompasses the case of “consisting of”. Additional objects, advantages and features of the invention will become apparent to those skilled in the art upon examination of the description or may be learned by practice of the invention. The following examples and drawings are provided by way of illustration, and they are not intended to be limiting of the present invention. Furthermore, the present invention covers all possible combinations of particular and preferred embodiments described herein.

### EXAMPLES

### 1. Materials

Thermoplastic Collagen commercial grades Ekomat CF10 and CF30 were provided by Ekolber SL. Ekomat collagen is prepared according to the process described in EP2727938A1.
Bovine collagen powder (Kapro B95 SF supplied by DCP, the Netherlands), with protein content≥95%, fat content≤3% and particle size 95%<200 μm. It corresponds to the dry collagen powder precursor disclosed in WO2007104322 (Naturin).
Food grade bovine hide gelatin was provided by Gelita with a bloom grade 220 and mesh 18.
Glycerin 99.5% purity was provided by Tata Genaro.
Polycaprolactone (PCL) pellets PBI 012 by Natureplast, Mw 50,000, Melt Flow Rate (ISO 1133, 160° C., 2.16 kg) 7 g/10 min and melting point 58° C.
Poly(butylene succinate-co-butylene adipate) (PBSA) pellets PBE 001 by Natureplast Melt Flow Rate (ISO 1133, 160° C., 2.16 kg)=4-5 g/10 min, melting point 88° C. Polylactic Acid (PLA) pellets Ingeo 4043D produced by Natureworks, Melt Flow Rate (ASTM D1238, 210° C., 2.16 kg) 6 g/10 min, peak melt temperature 145-160° C.
Ethylene vinyl acetate grafted with maleic anhydride EVA-g-MAH Bynel 3810 produced by Dupont, Melt Flow Rate (ASTM D1238, 190° C., 2.16 kg) 2.6 g/10 min, melting point 75° C.

### 2. General Procedures for the Preparation of Blends Containing Collagen or Gelatin, and a Biodegradable Polyester

### Procedure 1. Preparation of Thermoplastic Blends Containing Collagen and a Biodegradable Polyester

Commercial Ekomat grades (compositions shown in table 1) and biodegradable polyesters both in pellet shape were fed at different ratios as shown in table 3 and melt blended into a co-rotating Twin-Screw-Extruder, Leistritz GL27: D=27 mm, L/D=36 at 100 rpm with a setting extrusion temperature from feeding zone to die head 50° C. to 100° C. at 4 kg/h to obtain at die exit two strands of 3 mm diameter which were cooled before being pelletized. A comparative blend CFO was prepared analogously.

### Procedure 2. Preparation of Thermoplastic Blends Containing Collagen and a Biodegradable Polyester

Thermoplastic collagen according to Naturin were produced by premixing collagen powder (Kapro B95 SF), water and glycerin in a laboratory z-blade mixer during 5 minutes at 100 rpm. The obtained compositions (shown in table 2) and biodegradable polyester pellets were fed in different ratios and melt blended as shown in table 3 into a co-rotating Twin-Screw-Extruder, Leistritz GL27: D=27 mm, L/D=36 at 50 rpm with a setting extrusion temperature from feeding zone to die head 50° C. to 100° C. at 4 kg/h throughput to obtain at die exit two strands of 3 mm diameter which were cooled before being pelletized.

### Procedure 3. Preparation of Comparative Blends Containing Thermoplastic Gelatin and Biodegradable Polyesters

Thermoplastic gelatin (tGel) was produced by premixing gelatin and glycerin in a weight ratio of 80% and 20% respectively of total mixed weight in a laboratory z-blade mixer during 5 minutes at 100 rpm. The mixed material and biodegradable polyester were fed in different ratios and melt blended as shown in table 3 into a co-rotating Twin-Screw-Extruder, Leistritz GL27: D=27 mm, L/D=36 at 50 rpm with a setting extrusion temperature from feeding zone to die head 50° C. to 100° C. at 4 kg/h throughput to obtain at die exit two strands of 3 mm diameter which were cooled before being pelletized.

TABLE 1
Thermoplastic collagen Ekomat (before
melt blending with polyester)

| Thermoplastic collagen | CF30 | CF10 | CF0 |
|---|---|---|---|
| Collagen (% by weight) | 60 | 64 | 65 |
| Glycerin (% by weight) | 18 | 7 | 0 |
| Water (% by weight) | 22 | 29 | 35 |

TABLE 2
Thermoplastic collagen Naturin (before
melt blending with polyester)

| Thermoplastic collagen | ET-G15W35 |
|---|---|
| Collagen (% by weight) | 50 |
| Glycerin (% by weight) | 15 |
| Water (% by weight) | 35 |

Table 3 shows the composition of compounds prepared by melt blending a component “a” (either thermoplastic collagen Ekomat, thermoplastic collagen Naturin, or thermoplastic gelatin (tGel)) and a component “b” (biodegradable polyester), where a: b is the weight ratio between “a” and “b”. Compound blend is designated as a+b.

TABLE 3

| | Weight ratio a:b | | | | |
|---|---|---|---|---|---|
| Thermoplastic blends | 90:10 | 70:30 | 50:50 | 30:70 | 10:90 |
| CF30 + PCL | X | X | X | X | X |
| CF30 + PBSA | X | X | X | X | X |
| CF10 + PBSA | | X | | X | |
| CF10 + PCL | | | | X | |
| ET-G15W35 + PCL | | X | X | X | |
| ET-G15W35 + PBSA | | X | | X | |
| tGel + PCL | | X | X | X | |
| CF0 + PCL | | | X | | |

### 3. Procurement of Blown Film Samples

Mono layer and three layer blown film samples were produced by means of a blown film extrusion line LF 250 By Labtech with one or three single screw extruders, diameter 20 mm, L/D 30. For compound blends comprising PCL, extruders temperature profile was set, from feeding extruder zone to metering extruder zone, from 50 to 100° C., keeping die head temperature at 100° C. For compounding blends containing PBSA extruder temperatures were set, from feeding extruder zone to metering extruder zone to 50 to 110 or 120° C., keeping die head temperature at 110° C. to 120° C. Film sample extrusion were performed after 3 weeks of compound samples were produced.

### 4. Tests and Evaluation

i. Water solubility
ii. Homogeneity of extruded films from compound blends
iii. Thermo-processability-blown film extrusion
iv. Melt Flow Rate (MFR)
v. Tensile stress and strain at break and water swelling of films
vi. Oxygen Transmission Rate (OTR)
vii. Water Vapor Transmission Rate (WVTR)
viii. Cell proliferation and cytotoxicity
ix. 3d Printability
x Mono oriented Monofilament
xi. Thermo-processability (Injection molding, 3D printing blown film extrusion)
   i. Water Solubility

### Sample Preparation for Water Solubility Test

Samples were produced by feeding the materials in ratios as shown in table 4 to an internal mixer by Brabender. The mixing conditions were 90° C., 60 rpm and 5 minutes after loading the ingredients and stabilizing the torque. After mixing the blended compound was collected and sheets of 1 mm were produced by means of a laboratory platen press by Dr Collin at 95° C. The sheets were cut into small pieces of approx. 0.5-2 cm in width and length.

### Evaluation Test Procedure

3 to 5 grams of the above-prepared samples were weighed on analytical balance and dried in a vacuum oven at 160° C. until weight was constant. The initial dry weights (Wᵢ) of the samples were recorded with 4 decimal. Samples were soaked into 100 mL of distilled water with manual or orbital stirred in a thermostated bath for 1 hour at 60° C. The samples containing gelatin were completely solubilized while the samples containing collagen were only partially solubilized. In the case of the latter, the insoluble part was separated with the help of a strainer, collected and dried in a vacuum oven at 160° C. until dry weight. The final dry weights (Wᵢ) were recorded with 4 decimal. Calculations on the water solubility were made taking into account initial weight, initial moisture and dry weight to know the percentage of protein that had been dissolved according to the following formula:

Water solubility (%)=(Wᵢ−W_{f})/Wᵢ*100

wherein Wᵢ is the dry weight of the sample (dried in a vacuum oven at 160° C. until constant weight) before soaking it in water, and W_{f} is the dry weight of the sample (dried in a vacuum oven at 160° C. until constant weight) after soaking it in water. The following results were obtained:

TABLE 4
Water solubility (%)

Weight ratio a:b

| | 70:30 | 50:50 | 30:70 | 100:0 |
|---|---|---|---|---|
| CF30 + PCL | 18% | 9% | 2% | 80% |
| tGel + PCL | 70% | 38% | — | 100% |

ii and iii. Homogeneity and Processability of Extruded Blown Films

Pellet samples having the composition indicated in tables 5 and 6 were prepared as described in procedures 1, 2 and 3 above. Films were extruded as detailed in section 3 above. Roughness measurements (Ra, Rz values, at least 10 measurements in longitudinal direction) were carried out according to ISO 4287 test standard with a Brukner profilometer model DektakXT.

TABLE 5
Homogeneity and processability of extruded blown films

Weight ratio a:b

| | 70:30 | 50:50 | 30:70 | 100:0 |
|---|---|---|---|---|
| CF30 + PCL | ◯ | ◯ | ◯ | — |
| tGel + PCL | ◯ | ◯ | — | — |
| CF30¹ | — | — | — | ▴ |
| CF30² | — | — | — | X |

X Not possible to extrude
▴ Extrudable but films show inhomogeneity in case Ekomat CF30 with high thickness variation and extremely high film tackiness
◯ Extrusion with no remarkable issues and homogeneous films (with no visible granules)
¹CF30 produced by Ekolber SL 3 days before film extrusion
²CF30 produced by Ekolber SL 3 weeks before film extrusion

TABLE 6
Roughness values of extruded blown films

| | Ra (μm) | Rz (μm) |
|---|---|---|
| CF30 + PCL (50:50) | 2 | 8 |
| CF30 + PCL (70:30) | 2 | 9 |
| ET-G15W35 + PBSA (70:30) | 9 | — |
| ET-G15W35 + PCL (50:50) | 9 | — |

Rz Average amplitude - represents the average of the maximum peak-to-valley distances obtained at each of the basic lengths into which the measurement length is divided.
Ra Arithmetic Mean Roughness: it is the arithmetic average of the deviations of the roughness profile from the centre line along the evaluation length lm.

Results make evident that compositions according to invention (CF30+PCL at 70:30, 50:50, or 30:70) provided a significant reduction of water solubility compared to Ekomat grade CF30 and to blend compounds comprising thermoplastic gelatin, and, at the same time, they provide monophasic films with no visible granules in the film (not shown in the results).

Blown film extrusion of CF30 pellets produced by Ekolber 3 weeks before extrusion was not possible since melt material barely flowed in the extruder at setting processing temperatures. Additionally, it was found that the blend containing collagen, water and PCL without glycerin, CFO, was not thermally processable by film extrusion after a few days from its preparation (data not shown in the table).

iv. Melt Flow Rate (MFR)

An internal method based on ISO1133 was followed. 3-5 grams of pellet sample were charged into the cylinder of an apparatus for measuring MFR, sample was manually compressed with a rod and piston was placed into the cylinder assuring it had been at the testing temperature for at least 15 minutes before charging the sample. In case testing temperature was 90° C., sample remained in the cylinder for 5 minutes, preheating time, before testing load was placed on the piston and test started. When testing temperature was 100° C. or higher preheating time was one minute. Extrudate flowing from tester die was cut off and weighed every 2 minutes for 10 minutes. At least three measurements were done discarding the extrudate samples showing air bubble. All (pellet) samples were packed into sealed polyethylene bags at 23±2° C. before being tested after 3, 7 and 23 days from their production day.

Tables 7 to 10 below show the MFR values as measured at time points 0, 3 days, 7 days, and 23 days for blends of CF30+PCL, CF30+PBSA and CF10+PBSA, as well as the MFR difference (in g/10 min and in %) at 7 days and 23 days with respect to the initial MFR value. For comparison, MFR values for thermoplastic collagens CF30 and CF10, were also measured. Additionally, table 10 shows MFR test data for CF10 pellet sample stored at −18° C. and at room temperature after 3, 7 and 23 days

TABLE 7

| | | MFR (g/10 min) | | | | | |
|---|---|---|---|---|---|---|---|
| T | 90° C. | | CF30 + PCL (a:b) | | | | |
| Load | 9.6 kg | CF30 | 90:10 | 70:30 | 50:50 | 30:70 | 10:90 |
| Days | 0 | 48 | 34 | 32 | 36 | 24 | 15 |
| | 3 | 34 | 27 | 33 | 37 | 26 | 16 |
| | 7 | 5 | 24 | 28 | 33 | 22 | 13 |
| | 23 | 1 | 14 | 20 | 24 | 17 | 11 |

| | | | | | | |
|---|---|---|---|---|---|---|
| Variation MFR at 7 | −43 | −10 | −4 | −3 | −2 | −2 |
| days (in g/10 min) | | | | | | |
| Variation MFR at 7 | −90% | −29% | −9% | −8% | −8% | −13% |
| days (in %) | | | | | | |
| Variation MFR at 23 | −47 | −20 | −12 | −12 | −7 | −4 |
| days (in g/10 min) | | | | | | |
| Variation MFR at 23 | −98% | −59% | −37% | −33% | −29% | −27% |
| days (in %) | | | | | | |

TABLE 8

| | | MFR (g/10 min) | | | | | |
|---|---|---|---|---|---|---|---|
| T | 120° C. | CF30 | CF30 + PBSA (a:b) | | | | |
| Load | 9.6 kg | (90° C.) | 90:10 | 70:30 | 50:50 | 30:70 | 10:90 |
| Days | 0 | 48 | 118 | 85 | 70 | 35 | 13 |
| | 3 | 34 | 105 | 80 | 67 | 36 | 13 |
| | 7 | 5 | 80 | 75 | 64 | 34 | 12 |
| | 23 | 1 | 65 | 65 | 47 | 26 | 10 |

| | | | | | | |
|---|---|---|---|---|---|---|
| Variation MFR at 7 | −43 | −38 | −10 | −6 | −1 | −1 |
| days (in g/10 min) | | | | | | |
| Variation MFR at 7 | −90% | −32% | −12% | −9% | −3% | −8% |
| days (in %) | | | | | | |
| Variation MFR at 23 | −47 | −53 | −20 | −23 | −9 | −3 |
| days (in g/10 min) | | | | | | |
| Variation MFR at 23 | −98% | −45% | −24% | −33% | −26% | −23% |
| days (in %) | | | | | | |

TABLE 9

| | | MFR (g/10 min) | | |
|---|---|---|---|---|
| | T | | CF10 + PBSA (a:b) | |
| | 90° C. | | | 30:70 |
| Load | 9.6 kg | CF10 | 30:70 | (125° C.) |
| Days | 0 | 30 | 5 | 19 |
| | 3 | 21 | 4 | 14 |
| | 7 | 2 | 3 | 15 |
| | 23 | 0.5 | 3 | 12 |

| | | | |
|---|---|---|---|
| Variation MFR at 7 days (in g/10 min) | −28 | −2 | −4 |
| Variation MFR at 7 days (in %) | −93% | −40% | −21% |
| Variation MFR at 23 days (in g/10 min) | −29.5 | −2 | −7 |
| Variation MFR at 23 days (in %) | −98% | −40% | −37% |

TABLE 10

| Testing | | MFR, g/10 min CF10 | |
|---|---|---|---|
| (90° C. Load 2.16 kg) | | Storage 23° C. | Storage −18° C. |
| Days | 0 | 1.8 | 1.8 |
| | 3 | 1 | 1.9 |
| | 7 | 0.1 | 1.4 |
| | 23 | 0.1 | 1.1 |

| | | |
|---|---|---|
| Variation MFR at 7 days | −1.7 | −0.4 |
| Variation MFR at 7 days (in %) | −94% | −22% |
| Variation MFR at 23 days | −1.7 | −0.7 |
| (in g/10 min) | | |
| Variation MFR at 23 days (in %) | −94% | −39% |

### Results

Above tables reveal that after 23 days from supplier production date, Ekomat CF30 and CF10 barely flowed, did not flow homogenously and showed very irregular surface (data not shown in the tables). Also compound samples containing 10% of PCL or PBSA showed similar issue and a remarkable melt flowable decrease after 7 and 23 days. In contrast compound samples according to the invention showed less pronounced MFR decrease after 23 days from compound sample production.

Melt flow rate test results are coherent with the fact Ekomat and compounds containing Ekomat and 10% of PCL or PBSA, stored at room temperature for 21 days, could not be extruded for producing blown film samples while compounds samples containing Ekomat and at least 30% of PCL or PBSA could.

Table 10 shows MFR values of Ekomat CF10 pellets stored at 23° C. and −18° C. Storage of Ekomat under frozen conditions minimize melt flow decrease exhibited by same pellets stored at room temperature evidencing the influence of storage temperature on melt flow evolution. This fact reveals that industrial management of collagen thermoplastic pellets can be limited and problematic since Ekomat grades require to be stored at freezing conditions or, in case to be stored at room temperature, to be thermomoulded within a very short time since Ekomat production date.

In two additional MFR tests, and in order to increase Ekomat CF10 melt flow, testing temperatures were set at 125° C. and 110° C. However, this led to bubble formation into CF10 extrudate due to water vaporisation and consequently it was not possible to obtain any melt flow rate data. In contrast, CF10+PBSA 30:70 tested at increased temperature provided smooth, homogeneous, and bubble-free extrudates.

### CONCLUSION

Test results showed that compounds according to the invention comprising blends of Ekomat and at least 30% of PCL or PBSA provided higher melt flow stability during storage at room temperature than comparative thermoplastic collagen samples not blended with polyester compounds. Moreover, molten Ekomat barely flowed, or it flowed irregularly, after 7 days and particularly 23 days after being produced when stored at room temperature. Increasing testing temperature or increasing the water content could increase Ekomat MFR, however that led to water vaporisation and bad quality of extrudate.

v. Blown Film Mechanical Properties (Tensile Stress-Strain) and Water Swelling

Film samples (monolayer films having the compositions shown below and prepared as described in section 3) were tested after 7 days after extrusion. 48 hours before testing the samples were conditioned and kept at controlled temperature, 23±2° C., and 45±5% or 100% relative humidity (RH). Test was performed according to ISO 527-3 for determining tensile strength and elongation at break of film samples in machine direction (MD) and transverse direction (TD). Swelling into water is determined by weight difference of film samples before and after soaking them into water for 4 hours at 23±2° C. Swelling is expressed as the % and it is determined as (W1−W2/W1)*100, where W1 and W2 are the weights of the sample before and after being soaked into water, respectively.

TABLE 11
Monolayer film tensile properties

| | Tensile strength | | Tensile elongation | | Film |
|---|---|---|---|---|---|
| Films (conditioning: 48 h at | at break (MPa) | | at break (MPa) | | thickness |
| 23 ± 2° C., 45 ± 5% RH) | MD | TD | MD | TD | (μm) |
| CF30 + PBSA (70:30) | 44 | 26 | 21 | 15 | 67 |
| CF10 + PBSA (30:70) | 16 | 9 | 40 | 56 | 69 |
| CF30 + PCL (30:70) | 14 | 8 | 418 | 94 | 74 |
| CF10 | 39 | 21 | 2 | 2 | 131 |
| CF30 | 31 | 12 | 3 | 2 | 119 |
| ET-G15W35 | 15 | 10 | 4 | 3 | 190 |
| ET-G15W35 + PBSA (70:30) | 3 | 3 | 9 | 24 | 195 |
| ET-G15W35 + PBSA (30:70) | 4 | 4 | 42 | 44 | 115 |
| ET-G15W35 + PCL (50:50) | 4 | 3 | 51 | 28 | 195 |
| ET-G15W35 + PCL (30:70) | 4 | 2 | 36 | 21 | 130 |

| | |
|---|---|
| CF30 + PLA (50:50) | It was not possible to extrude any film |

The swelling of CF30+PCL (30:70) (4 h in water 23° C.) was 34%.

### Results

Monolayer blown films extrusion using ET-G15W35 thermoplastic collagen were problematic and the quality of sample was not acceptable. Films had uneven thickness, with very rough surface and become very brittle after 24 or 48 hours.

Monolayer films comprising Ekomat CF10 and CF30, where Ekomat pellets were stored at −18° C. before extrusion to avoid loss of their melt flowability, although being optically monophasic, became very brittle after one day and additionally showed high water solubility (table 4, CF30+PCL 100:0).

By contrast, thermoplastic blend films comprising Ekomat and polyester according to the invention provided better extrudability, mechanical properties, exhibiting less brittleness and higher values of elongation at break.

In addition, they showed significant lower water solubility than monolayers films comprising CF10 and CF30 (table 4 CF30+PCL blends 70:30, 50:50, 30:70) they provided better resistance to high % RH atmosphere (% 100, data not shown in the table).

Monolayer film extrusion of blends 50:50 in weight PLA (having a melting point around 145-160° C.) and CF30 (i.e., outside the scope of the claims) was not possible since required minimum processing temperature to process the blend leads to bubble and foaming of the extrudate due to vaporisation of water. Temperature profile of extruder at die was set as low as 170° C. to melt PLA material.

vi. Oxygen Transmission Rate (OTR) of Blown Film Samples

Film samples oxygen transmission rate (OTR) was analyzed in accordance with DIN 53 380 Standard Test Method at 23° C. and 50% relative humidity by means of an Ox-Tran® permeation analyzer by Mocon.

TABLE 12

| | OTR (cm³/m² · day) | Film thickness (μm) |
|---|---|---|
| CF30 | 3 | 155 |
| CF30 + PCL (30:70) | 570 | 105 |

Films according to the invention showed as expected lower oxygen barrier (i.e. higher OTR values) than films based on thermoplastic collagen. Besides, they provided better (higher) oxygen barrier than low density polyethylene films (data not shown).

vii. Water Vapor Transmission Rate (WVTR) of Blown Film Samples

Water vapor transmission rate was determined in accordance with DIN 53-122 Standard Test Method at 23° C. and 85% relative humidity by means of a Permatran-W® permeation analyzer by Mocon. Samples were conditioned for 48 h at 23° C. 50% RH before testing.

TABLE 13

| | WVTR (g/m² · day) | Film thickness (μm) |
|---|---|---|
| CF30 | 1162 | 155 |
| CF30 + PCL (30:70) | 765 | 105 |

The film according to the invention improved the water vapor barrier properties in more than 30% compared to collagen film without PCL.

viii. Cell Proliferation and Cytotoxicity

The cytotoxicity of films prepared from blends according to the invention was assessed and compared to other films. The following films were tested:
Film 1: Collagen Cell Carrier® membrane (CCC) produced by Viscofan
Film 2: CF30+PCL (30:70)
Film 3: CF30+PCL (70:30)
Film 4: 100% PCL

Films 2-3 were prepared by thermo-pressing collagen or collagen+PCL in pellet form by means of a laboratory heated platen press to obtain plates of approx. 1 mm thickness.

Human dermal fibroblasts were seeded on the films, previously sterilized at 25 KGy (Gamma irradiation). As a positive control (C+), cells were seeded onto standard commercial polystyrene well plates treated for optimal cell attachment (Corning ref 3595). As a negative control, not treated commercial polystyrene well plates (Corning ref 351178) were used. For seeding on films, a cell suspension was prepared and a 50 μl drop was deposited on each film, thus obtaining a final density of 14,000 cells/cm². The samples were placed in 24-well plates and incubated for 3 h at 37° C. and 5% CO₂. Next, they were immersed in 1 mL of complete medium. After 24 h of incubation, 4 replicates of each sample were analyzed by WST-1 and 2 replicates by Live/Dead. The WST-1 assay was performed with the commercial kit “Cell Proliferation Reagent WST-1” from Roche Applied Science. The films were incubated in 300 μl of a serum-free WST-1 solution for 4 h at 37° C. and 5% CO₂, after which 100 μl of each sample was extracted and analyzed for absorbance at 440 nm using a plate spectrophotometer (Sinergy HT, Biotek).

For the Live/Dead assay, samples were washed with saline buffer and incubated for 20 min in calcein solution at 37° C. and 5% CO₂. The solution was removed and dead cells were stained with propidium iodide solution for 5 min. The samples were washed with a saline buffer and fluorescence images were acquired by means of a confocal microscope (Stellaris 5, Leica Geosystems, Switzerland).

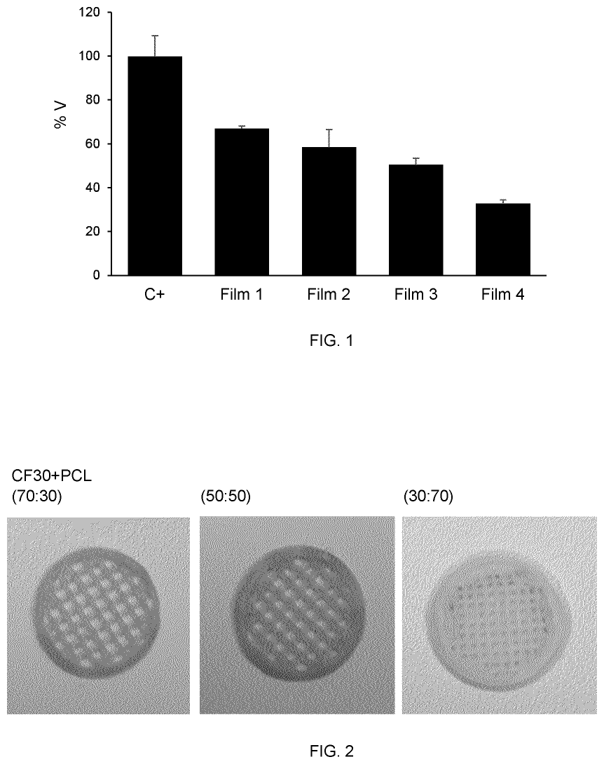

As shown in FIG. 1 the cell viability in the films showed significant differences between the different types of samples. Films 1 (Collagen Cell Carrier® membrane (CCC) produced by Viscofan) and 2 (CF30+PCL (30:70))) presented the highest viability, indicating that these are the samples with the highest cell adhesion. A small decrease in cell viability was observed in film 2, however, not to significant values. Film 3 (CF30+PCL (70:30))) presented viability values of 50%, slightly lower compared to films 1 and 2. Finally, film 4 (100% PCL) and negative control (data not shown), showing both same value, exhibited a significant decrease in cell viability. None of the films showed signs of toxicity.

### ix. 3D Printability

The following pellet samples were used:
Ekomat CF30+PCL (70:30)
Ekomat CF30+PCL (50:50)
Ekomat CF30+PCL (30:70)

For the printing of these materials, a domoBIO 2A (Domotek, Spain) printer was used, with the configuration of the pellet extruder head (Mahor V4) and a high-performance heated bed. In addition, a Buildtack printing surface to ensure the adhesion of the material was used. Due to the low melting point of the material, the hopper fan was kept activated to prevent the material from melting before entering the extruder screw and blocking it. The parameters used for impressions were as follows:

### Parameter Value Unit

Head of extrusion 0.8 mm
Layer height 0.2 mm
Layer width 0.8 mm
Infill overlap 80%
Extruder Temperature 90° C.
Platform Temperature 40° C.
Printing Flow 250%
Printing speed 3 mm/s
Retraction distance 6.5 mm
Retraction speed 25 mm/s

FIG. 2 shows the printed materials with the blends according to the invention. All the materials containing compounds according to the invention were printable under the described conditions. By contrast, Ekomat CF30 could not be printed out. The extrudate was very irregular with no homogeneous feeding.

x. Monofilament Extrusion

As described in procedure 1, Ekomat CF30 grade and PCL both in pellet form were fed in a weight ratio of 1:1 and melt blended in a co-rotating Twin-Screw-Extruder, Leistritz GL27: D=27 mm, L/D=36 at 150 rpm with a setting extrusion temperature from feeding zone to die head 70° C. to 90° C. at 6 kg/h to obtain at die exit two strands of 3 mm diameter which were cooled before winding them continuously for having filament reels.

After 24 hour filament reel were unwind and mono-axially stretched by means of a Teach Line MDO-AT by Collin. Drawing process consists in passing the preheated filaments at 45° C. over a series of rollers. The rollers rotate at specified constant angular velocities, each one faster than the previous. The higher rollers speed difference, the higher is monofilament stretching. Orientation ratio is defined herein as v2/v1 where v1 and v2 are the velocities of firsts rollers and subsequent rollers in m/min.

TABLE 14

| | Orientation | Tensile strength | Tensile elongation |
|---|---|---|---|
| Samples | ratio | (MPa) | (%) |
| CF30 + PCL (50:50) | Non oriented | 14 | >500 |
| | 4 | 112 | 126 |
| | 5 | 79 | 116 |

*Orientation ratio: speed (m/min) ratio of second (pulling) nip roll:first nip roll

According to the obtained results, the monofilament comprising Ekomat and PLC according to the invention showed significant tensile strength which increased when stretched.

xi. Thermo Processability (Blown Film Extrusion, Injection Moulding, 3D Printing) As shown in the table below, compound blends of Ekomat CF10 or CF30 and PCL or PBSA in the ratios 70:30 to 10:90 are more easily thermo processed while high content Ekomat (90:10) led to very difficult process or it is not industrially feasible.

TABLE 15

| Samples | Thermomoulding |
|---|---|
| CF30 or CF10 + | Narrow temperature must be lower than 120° C. (water |
| PCL or PBSA | sensibility) |
| (90:10) | Extremely sticky - not possible to produce quality blown film |
| | Degradation in extruder/injection when increasing residence |
| | time |
| | Variation of viscosity melt through the extrusion die |
| | 3D print no stable melt flow/extrudate - not feasible to print |
| CF30 or CF10 + | Broader processability temperature window |
| PCL or PBSA | No sticky film/easy demoulding of the injected moulded parts |
| (70:30-10:90) | Stable viscosity melt through the extrusion die |
| | 3D print stable melt flow/extrudate - feasible to print |

### CITATION LIST

EP2727938
WO2007104322
EP2596051B1
ISO 1133-3 (Third edition, 1997 Jan. 15). Plastics—Determination of the melt mass-flow rate (MFR) and the melt volume-flow rate (MVR) of thermoplastics
ISO 527-3 (First edition, 1995 Aug. 1). Plastics—Determination of tensile properties—Part 3: Test conditions for films and sheets
ISO 4287 (First edition Premiere edition 1997 Apr. 1). Geometrical Product Specifications (GPS)—Surface texture: Profile method—Terms, definitions and surface texture parameters

For reasons of completeness, various aspects of the invention are set out in the following numbered clauses:

Clause 1. A blend composition comprising:
i) collagen;
ii) water;
iii) particularly a plasticizer; and
iv) a polyester having a melting point equal to or lower than 120° C.;
wherein the weight ratio of the collagen and the polyester is from 2:1 to 1:20.

Clause 2. The blend composition according to clause 1, which is meltable.

Clause 3. The blend composition according to any of the clauses 1-2, which is in a solid state at a temperature equal to or lower than 40° C. or alternatively at room temperature (20-25° C.), more particularly in the form of solid pellets.

Clause 4. The blend composition according to any of the clauses 1-3, which is able to be thermoformed, in particular by melt blending the collagen-based blend composition as defined herein; and extruding, casting or blowing the blend of step a) into a solid article.

Clause 5. The blend composition according to any of the clauses 1-4, which is able to provide a homogeneous film when submitted to blow film extrusion.

Clause 6. The blend composition according to any of the clauses 1-5, which is able to provide a sheet having a water solubility equal to or lower than 40% w/w.

Clause 7. The blend composition according to any of the clauses 1-6, wherein the collagen is partially denatured.

Clause 8. The blend composition according to any of the clauses 1-7, wherein the polyester is selected from the group consisting of polycaprolactone (PCL), polybutylene succinate-co-adipate (PBSA), polyethyleneadipate (PEA), polybutylenesuccinate (PBS), polybutylene adipate terephthalate (PBAT), and combinations thereof.

Clause 9. The blend composition according to clause 8, wherein the polyester is polycaprolactone (PCL) or polybutylene succinate-co-adipate (PBSA).

Clause 10. The blend composition according to any of the clauses 1-9, wherein water is present in an amount from 5 to 40%, by weight with respect to the total weight of the blend composition.

Clause 11. The blend composition according to any of the clauses 1-10, wherein the weight ratio of the collagen and the polyester is from 1.9:1 to 1:18.

Clause 12. The blend composition according to any of the clauses 1-11, which comprises a plasticiser.

Clause 13. The blend composition according to clause 12, wherein the plasticiser is selected from the group consisting of glycerin, ethylene glycol, a tri(C₃-C₁₂)alkylester of glycerin, a sulfated fatty acid, lecithin, and a combination thereof.

Clause 14. The blend composition according to any of the clauses 12-13, wherein the weight ratio of the collagen and the plasticizer is from 100:5 to 100:60.

Clause 15. The blend composition according to any of the clauses 1-14, which further comprises soy protein or gluten.

Clause 16. A solid shaped article which is obtainable by a process comprising:
a) melt blending the blend composition as defined in any of the clauses 1-15; and
b) extruding, casting or blowing the blend of step a) into a solid article.

Clause 17. The solid shaped article according to clause 16, which is selected from the group consisting of a film, an injection moulding article, a blow moulding article, a monofilament, a mono-oriented filament, an extrusion blown bottle, a 3D printed object, an injection molded object or a machined piece, and a filament for 3D printing by Fused Deposition Modelling (FDM).

Clause 18. The solid shaped article according to clause 17, which is a film selected from the group consisting of a monolayer film and a multilayer film.

Clause 19. The solid shaped article according to clause 18, which has one or more of the following properties:
a) a melt flow rate variation at 90° C. and load of 9.6 kg is equal to or lower than −50% after 7 days;
b) a melt flow rate variation at 120° C. and load of 9.6 kg is equal to or lower than −50 after 23 days;
c) a melt flow rate variation at 90° C. and load of 9.6 kg is from equal to or lower than −30% after 7 days;
d) a melt flow rate variation at 120° C. and load of 9.6 kg is from equal to or lower than −40% after 23 days;
e) an Oxygen Transmission Rate (OTR) equal to or lower than 600 cm³/m²·day, at 23° C. and 50% relative humidity;
f) a Water Vapor Transmission Rate (WVTR) equal to or lower than 800 g/m²·day, at 23° C. and 85% relative humidity;
g) a tensile strength at break in machine direction from 3 to 50 mPa;
h) a tensile strength at break in transverse direction from 3 to 30 mPa;
i) a tensile elongation at break in machine direction from 9 to 450%; and
j) a tensile elongation at break in transverse direction from 10 to 150%.

Clause 20. Use of the blend composition as defined in any of the clauses 1-15 as support for in vitro assays.

Clause 21. Use of the blend composition as defined in any of the clauses 1-15 or the solid shaped article as defined in any of the clauses 16-19, in the food industry, in the pet food industry, in the agricultural industry, or in the pharmaceutical industry.

What is claimed is:

1. A blend composition comprising:
i) collagen;
ii) water;
iii) a plasticizer; and
iv) a polyester having a melting point equal to or lower than 120° C.;
wherein the weight ratio of the collagen and the polyester is from 2:1 to 1:20.

2. The blend composition according to claim 1, which is in solid state at a temperature equal to or lower than 40° C. and is meltable.

3. The blend composition according to claim 1, wherein the polyester is selected from the group consisting of polycaprolactone (PCL), polybutylene succinate-co-adipate (PBSA), polyethyleneadipate (PEA), polybutylenesuccinate (PBS), polybutylene adipate terephthalate (PBAT), and combinations thereof.

4. The blend composition according to claim 3, wherein the polyester is polycaprolactone (PCL) or polybutylene succinate-co-adipate (PBSA).

5. The blend composition according to claim 1, wherein water is present in an amount from 5 to 40%, by weight with respect to the total weight of the blend composition.

6. The blend composition according to claim 1, wherein the weight ratio of the collagen and the polyester is from 1.9:1 to 1:18.

7. The blend composition according to claim 1, wherein the plasticiser is selected from the group consisting of glycerin, ethylene glycol, a tri(C₃-C₁₂)alkylester of glycerin, a sulfated fatty acid, lecithin, and a combination thereof.

8. The blend composition according to claim 6, wherein the weight ratio of the collagen and the plasticizer is from 100:5 to 100:60.

9. The blend composition according to claim 1, which further comprises soy protein or gluten.

10. A solid shaped article which is obtainable by a process comprising:
a) melt blending the blend composition as defined in claim 1; and
b) extruding, casting or blowing the blend of step a) into a solid article.

11. The solid shaped article according to claim 10, which is selected from the group consisting of a film, an injection moulding article, a blow moulding article, a monofilament, a mono-oriented filament, an extrusion blown bottle, a 3D printed object, an injection molded object or a machined piece, and a filament for 3D printing by Fused Deposition Modelling (FDM).

12. The solid shaped article according to claim 11, which is a film selected from the group consisting of a monolayer film and a multilayer film.

13. The solid shaped article according to claim 12, which has one or more of the following properties:
a) a melt flow rate variation at 90° C. and load of 9.6 kg is equal to or lower than −50% after 7 days;
b) a melt flow rate variation at 120° C. and load of 9.6 kg is equal to or lower than −50 after 23 days;
c) a melt flow rate variation at 90° C. and load of 9.6 kg is from equal to or lower than −30% after 7 days;
d) a melt flow rate variation at 120° C. and load of 9.6 kg is from equal to or lower than −40% after 23 days;
e) an Oxygen Transmission Rate (OTR) equal to or lower than 600 cm³/m²·day, at 23° C. and 50% relative humidity;
f) a Water Vapor Transmission Rate (WVTR) equal to or lower than 800 g/m²·day, at 23° C. and 85% relative humidity;
g) a tensile strength at break in machine direction from 3 to 50 mPa;
h) a tensile strength at break in transverse direction from 3 to 30 mPa;
i) a tensile elongation at break in machine direction from 9 to 450%; and
j) a tensile elongation at break in transverse direction from 10 to 150%.

14. A method of culturing cells in vitro comprising incubating the cells in the presence of the blend composition as defined in claim 1.

15. An article comprising the blend composition as defined in claim 1, wherein the article is selected from the group consisting of a food product, a pet food or pet food product, an agricultural product, and a pharmaceutical product.

16. An article comprising the solid shaped article as defined in claim 10, wherein the article is selected from the group consisting of a food product, a pet food or a pet food product, an agricultural product, and a pharmaceutical product.

17. The blend composition according to claim 1, wherein:
the polyester is selected from the group consisting of polycaprolactone (PCL), polybutylene succinate-co-adipate (PBSA), polyethyleneadipate (PEA), polybutylenesuccinate (PBS), polybutylene adipate terephthalate (PBAT), and combinations thereof; and
the plasticiser is selected from the group consisting of glycerin, ethylene glycol, a tri(C₃-C₁₂)alkylester of glycerin, a sulfated fatty acid, lecithin, and a combination thereof.

18. The blend composition according to claim 17, wherein
water is present in an amount from 5 to 40%, by weight with respect to the total weight of the blend composition.

19. The blend composition according to claim 3, wherein the weight ratio of the collagen and the polyester is from 1.9:1 to 1:18.

20. The blend composition according to claim 7, wherein the weight ratio of the collagen and the plasticizer is from 100:5 to 100:60.
